(19) United States
(12) Patent Application Publication
(10) Pub. No.: US 2007/0000802 A1
(43) Pub. Date: Jan. 4, 2007

(54) Holder for intervertebral prostheses

(75) Inventor: Arnold Keller, Kayhude (DE)

Correspondence Address: MORRISON & FOERSTER LLP, 1650 TYSONS BOULEVARD, SUITE 300, MCLEAN, VA, 22102, US

(73) Assignee: Cervitech, Inc., Rockaway, NJ (US)

(21) Appl. No.: 11/454,117

(22) Filed: Jun. 16, 2006

(30) Foreign Application Priority Data
Jun. 21, 2005 (DE) 20 2005 009 755.1

(51) Int. Cl. A61J 1/00; A61F 2/46

(52) U.S. Cl. 206/438

(57) ABSTRACT

An arrangement includes a holder for holding intervertebral prostheses, at least two intervertebral prostheses of different widths which each have two end plates with ventral flanges and a joint core arranged between the end plates. The holder includes a support plate with at least one opening formed therein that is configured to receive one of the intervertebral prostheses and has a width which is greater that the maximum width of the intervertebral prostheses. A pair of steps is formed on at least one longitudinal edge of the opening that has flanks that are directed toward one another and spaced apart such that the flange of the intervertebral prosthesis inserted into the opening is centered in the opening. The pair of steps can be configured as projections or depressions.

### FIELD OF THE INVENTION

The invention relates to an arrangement comprising a holder for intervertebral prostheses, and intervertebral prostheses of different widths which consist of two end plates with a ventral flange and, arranged between them, a joint core, the holder having a support plate with an opening which receives one of the intervertebral prostheses and whose width is greater than the maximum width of the intervertebral prostheses.

### BACKGROUND OF THE INVENTION

It is known to arrange intervertebral prostheses in a holder from which they are removed by the operating surgeon and implanted (EP-A-1 504 733). The intervertebral prostheses are removed from the holder by means of a special insertion instrument designed for implantation of the intervertebral prostheses. The intervertebral prosthesis and insertion instrument have projections and recesses which engage in one another to ensure positionally accurate fixing of the intervertebral prosthesis on the insertion instrument. So that the intervertebral prostheses can be received precisely by the insertion instrument, the intervertebral prostheses are arranged in a special holder. The latter is adapted exactly to the dimensions of the respective intervertebral prosthesis. Different holders are needed for intervertebral prostheses having different dimensions. For simple handling, the holders are often combined to form magazines that have receiving openings for intervertebral prostheses of different widths or heights. Although this has the advantage that the operating surgeon can be given a large choice of intervertebral prostheses during surgery, such a magazine is quite large and awkward to handle.

### SUMMARY OF THE INVENTION

The object of the invention is to make available an arrangement comprising an intervertebral prosthesis and a holder for intervertebral prostheses, permitting simpler handling, even in the case of intervertebral prostheses of different sizes.

The solution according to the invention lies in the features of the invention as disclosed broadly herein. Advantageous developments are the subject matter of the specific embodiments disclosed below.

According to the invention, a holder for intervertebral prostheses is provided, and also intervertebral prostheses of different widths which consist of two end plates with a ventral flange and, arranged between them, a joint core, the holder comprising a substructure and a support plate with at least one opening which is designed to receive one of the intervertebral prostheses and whose width is greater than the maximum width of the intervertebral prostheses, the invention being characterized in that a pair of steps is formed on at least one longitudinal edge of the opening, and their flanks directed toward one another are spaced apart such that the ventral flange of the intervertebral prosthesis inserted into the opening is centered.

The width of the opening in the holder according to the invention is dimensioned such that intervertebral prostheses with different widths can be inserted into the opening. This means it is no longer necessary to use a conventional magazine-type holder with rows of openings of different widths for different sizes of intervertebral prostheses. In order also to ensure that an intervertebral prosthesis of small width, inserted into the holder according to the invention, is fixed in a positionally accurate manner for the purpose of safe removal by the insertion instrument, the invention proposes a pair of steps arranged on the longitudinal edges. This pair of steps is designed such that the spacing between the mutually facing flanks is matched to the width of the ventral flange on the end plates of the intervertebral prostheses. Matched is understood here as meaning that the spacing corresponds to the width of the flange plus a possible slight clearance for easy insertion and removal. In the inserted state, therefore, the intervertebral prostheses are fixed in position via their ventral flange. This ensures that the intervertebral prostheses in the holder are centered in the opening, and, specifically, independently of their width. It suffices therefore that the holder has only a single opening, and, unlike the magazine-type holder known from the prior art, there is no longer any need for a plurality of openings for intervertebral prostheses of different widths. This permits a compact, space-saving and easy-to-handle design of the holder. Since, by virtue of the invention, variants for intervertebral prostheses of different widths are not required, the holder according to the invention is especially suitable for mass production. It can be designed as a disposable article, which affords advantages in particular in respect of postoperative handling and the fact that sterilization is no longer necessary after the operation.

The pair of steps is preferably designed as a pair of projections. This is not absolutely essential, however. Provision can also be made for the pair of steps to be formed by means of a depression running between them. This kind of recessed arrangement of the pair of steps affords the advantage that the opening, and the intervertebral prostheses inserted in it, can be closed off at the same level by a cover arranged on the support plate. This makes it possible to sterilize the intervertebral prosthesis in the preferably closed holder.

The intervertebral prostheses can have different heights. This is generally achieved by using joint cores of different thickness. Since, before surgery, the operating surgeon often does not know what height of intervertebral prosthesis will be needed, provision is advantageously made for the holder according to the invention to have a second opening of different height. In this way, the operating surgeon can be offered a greater choice using a just slightly larger holder. It is not necessary here for the height of the opening to correspond exactly to the height of the intervertebral prosthesis. A filler piece can also be provided to compensate for any difference in height.

The mutually facing flanks of the pair of steps can be designed perpendicular to the plane of the support plate. However, they are expediently inclined in relation to the perpendicular by an angle of approximately 5 to 15 degrees, preferably of approximately 10 degrees. This has the effect that the spacing between the flanks of the pair of steps tapers conically toward the support plate. This facilitates insertion of the intervertebral prostheses into the holder according to the invention. The conicity is in this case expediently chosen such that, at the narrowest point, the spacing corresponds exactly to the width of the flange. The inserted intervertebral prosthesis is thus subjected to an additional frictional force and in this way is secured against accidentally falling out of the opening.

On the sides of the openings, top parts for the support plate can be provided whose mutually facing side surfaces form a guide for an insertion instrument. Removal of the intervertebral prostheses by means of an instrument can be made easier in this way. The pairs of steps are preferably designed in one piece with the top parts.

The opening in the support plate can have a closed configuration at the bottom and can function as a substructure. The intervertebral prosthesis inserted into the holder is in this way protected against damage or dirt. In order also to permit closure of the front face in the direction of insertion and removal, a cover can expediently be provided. Adhesives are particularly suitable as the cover, or also sealed films. This permits sterile packing of the intervertebral prosthesis in the holder according to the invention.

The support plate and the substructure can be assembled from several parts, but they can also be designed in one piece. The latter is particularly recommended where the support plate and substructure are produced by thermoforming of a planar material, such as a thermoplastic. This ensures particularly cost-effective manufacture of the holder according to the invention. The holder can be designed as a disposable product.

### BRIEF DESCRIPTION OF THE DRAWINGS

The invention is explained below with reference to the drawings which depict advantageous illustrative embodiments and in which:

FIG. 1 shows a plan view of a first illustrative embodiment of the arrangement according to the invention with a holder and with an intervertebral prosthesis inserted therein;

FIG. 2 shows an enlarged front view of the holder depicted in FIG. 1;

FIG. 3 shows a bottom view of the holder depicted in FIG. 1;

FIG. 4 shows a perspective view and plan view of the intervertebral prosthesis;

FIG. 5 shows a plan view of a second illustrative embodiment of the arrangement according to the invention;

FIG. 6 shows a cross section along the line VI-VI in FIG. 5; and

FIG. 7 shows a third illustrative embodiment in a view similar to FIG. 6.

### DETAILED DESCRIPTION OF THE INVENTION

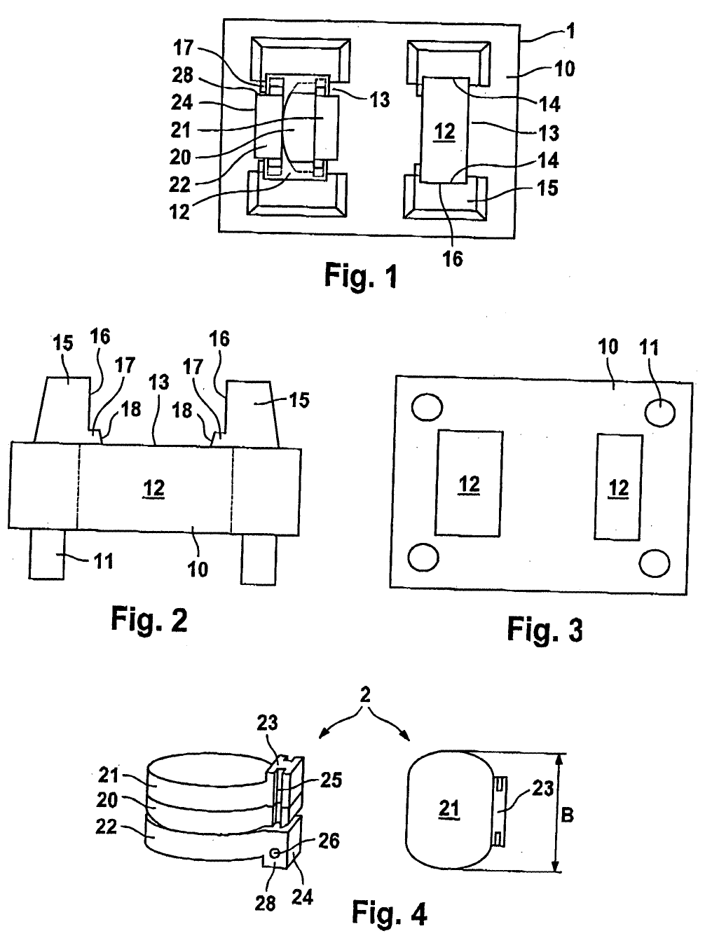

A first illustrative embodiment of the invention with a holder 1 and with an inserted intervertebral prosthesis 2 is shown in FIG. 1, the holder 1 is shown on its own in FIGS. 2 and 3, and the intervertebral prosthesis is shown on its own in FIG. 4.

The holder 1 comprises a support plate 10 made from a metal, for example stainless steel. This plate is of parallelepipedal configuration, and its dimensions are such that, in the illustrative embodiment shown, it is able to support two openings 12 for receiving the intervertebral prostheses 2. The support plate rests on a substructure 11, which is designed as four cylindrical feet.

The structure of the intervertebral prosthesis 2 to be received will now be explained. As is shown in FIG. 4, the intervertebral prosthesis 2 consists of two end plates 21, 22 and of a prosthesis core 20 arranged between these. The prosthesis core 20 forms, with the two end plates 21, 22, a joint which permits interlinking of the two end plates 21, 22. The dimensions of the prosthesis 2 can be varied. Vertically, this is achieved by prosthesis cores 20 of different thickness. Widthwise, this is achieved by end plates 21, 22 of different width B. The end plates 21, 22 each have a flange 23, 24, respectively, along their ventral edge (to the right in FIG. 4). On the side directed away from the prosthesis core and intended to bear on the vertebral bodies (not shown), the flange protrudes outward by a certain distance. The width of the flanges 23, 24 is smaller than the width B of the end plates 21 and is constant in intervertebral prostheses 2 of different width. The flange 23 has a continuous longitudinal groove 25 in its side surfaces. It is used for receiving a projecting ridge of an insertion instrument (not shown). The flange 24 has a bore 26 in each of its side surfaces 28. This bore 26 is used for receiving a pin of the insertion instrument. By means of the bore 26 and the slit 25, the intervertebral prosthesis 2 can be mounted with a form fit and in a defined position on the insertion instrument.

The holder 1 is provided for storing the intervertebral prosthesis prior to implantation and for passing it to the operating surgeon during the intervention. It comprises the two openings 12, in at least one of which an intervertebral prosthesis 2 is to be inserted. Each opening 12 is enclosed by longitudinal edges 13 and transverse edges 14. The two openings 12 have different dimensions in the direction of the height of the intervertebral prosthesis 2. This is used for taking up intervertebral prostheses 2 of different thickness. In FIG. 1, an intervertebral prosthesis 2 of considerable thickness is inserted into the opening 12 shown on the left in the drawing. The length of the transverse edges 14 correlates with the thickness of the intervertebral prosthesis 2 to be fitted, such that the intervertebral prosthesis is fixed in this direction. The width of the openings 12 is chosen such that it is at least as great as the maximum width B of the intervertebral prostheses 2 that are to be received. This ensures that the intervertebral prosthesis 2 can be inserted into the opening 12 independently of its width. In the inserted state, the end plates 21, 22 and prosthesis core 20 are submerged almost completely into the opening 12. The end plates 21, 22 lie only via their respective flange 23, 24 on the longitudinal edges 13 of the opening 12. An intervertebral prosthesis with a smaller width than the maximum width is, however, not fixed in the width direction by the dimensions of the opening 12. In order for the intervertebral prosthesis 2 inserted in the opening 12 to be definitively positioned, however, a pair of steps 17 is provided according to the invention. This pair of steps 17 comprises mutually facing flanks 18 which, in the illustrative embodiment shown, gently taper conically toward the support plate 10. The spacing of the mutually facing flanks 18 is adapted to the width of the flange 24. In the depicted conical shape of the flanks 15 of the pair of steps 17, this is achieved by the fact that the spacing at the transition of the flanks 18 to the support plate 10 corresponds exactly to the target dimension of the width of the flange 24. The intervertebral prosthesis 2 inserted into the opening 12 then sits tightly between the pair of steps 17. If, in order to permit easy removal, this is not desired, a slight play of a few tenths of millimeters, preferably of 0.1 to 0.7 mm, can also be provided. The conicity of the flanks 18 permits simplified insertion of the intervertebral prosthesis 2 into the opening 12 of the holder 1, specifically even in the case of a tight fit.

Guide parts 15 are arranged on the transverse edges 14 of the openings 12. On their inner faces directed toward one another, they have a guide channel 16 which is flush with the transverse edges 14 and with the respective ends of the longitudinal edges 13. Upon removal of the intervertebral prosthesis 2 from the holder 1, the guide channel 16 is used to guide the tip of the insertion instrument in such a way that its projections formed as pin and ridge engage in the slit-like depression 25 and in the bore 26 of the flanges 23, 24. Since, by virtue of the invention, the prosthesis is centered in the opening 12, the intervertebral prosthesis 2 is picked up by the insertion instrument quickly and without repositioning of the intervertebral prosthesis 2 or of the instrument.

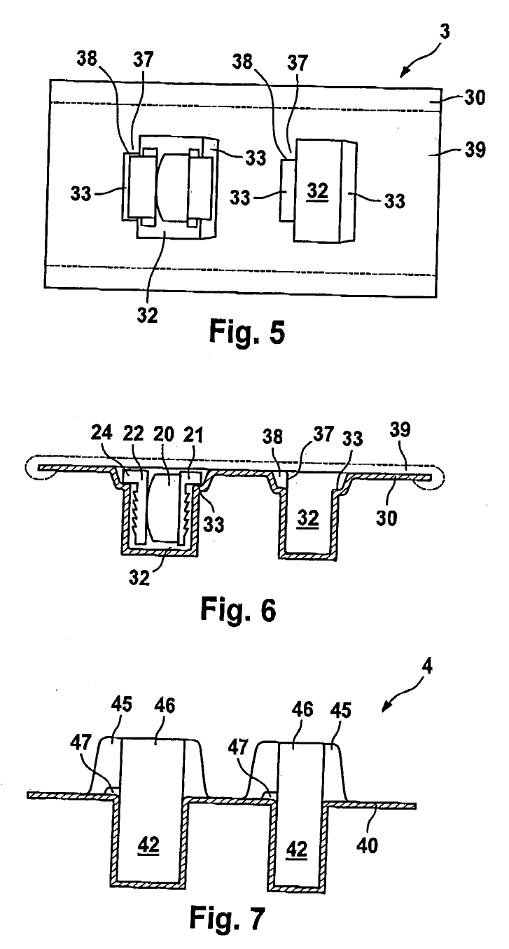

A second illustrative embodiment is shown in FIGS. 5 and 6. It mainly differs from the first illustrative embodiment shown in FIGS. 1 through 3 in that it is produced from a web-like material. This can, for example, be deep-drawn sheet steel or an autoclavable plastic. The web 30 of the holder 3 is shaped such that two pocket-like openings 32 are obtained. In contrast to the first illustrative embodiment, the openings 32 are closed at the bottom and at the side. A further difference from the first illustrative embodiment is that the longitudinal edges 33 are recessed. They are not flush with the upper plane of the web material 30, but instead are recessed by an amount corresponding approximately to the thickness of the flanges 23, 24. The depth of the openings 32 is dimensioned such that, despite the recessed longitudinal edges 33, the intervertebral prosthesis 2 can be received completely. A pair of steps 37 is arranged on at least one longitudinal edge 33 of each opening 32. In this area, the longitudinal edge 33 is not recessed. In the area of transition between the non-recessed and recessed parts of the longitudinal edge 33, the pair of steps 37 with its flanks 38 is formed. The flanks 38 have a spacing that corresponds to the width of the flange 24. As in the case of the first illustrative embodiment, this means that intervertebral prostheses with different widths B can be inserted into the openings 32, the side surfaces 28 of the flange 24 in this case coming into contact with the flanks 38 of the pair of steps, in order in this way to center the intervertebral prosthesis 2 in the opening 32.

In the second illustrative embodiment of the holder 3 according to the invention, sterile storage is permitted by means of the recessed arrangement of the intervertebral prosthesis 2. For this purpose, a cover film 39 is provided which extends across the upper part of the web material, functioning as support plate 30, from one end to the opposite end in such a way that the two openings 32 are covered. This not only permits effective and inexpensive construction of the holder 3 as a disposable product, but also simplified handling in which the intervertebral prosthesis 2 remains in the sterile holder until the moment of implantation.

FIG. 7 shows a third illustrative embodiment which represents a modification of the second illustrative embodiment shown in FIGS. 5 and 6. Both have in common the fact that the holder is produced from a web material. However, in the holder 4 shown in FIG. 7, the web material 40 is shaped such that closed depressions are obtained as openings 42, and also tower-like top parts 45 with a guide 46, and a pair of steps 47 for centering the intervertebral prosthesis 2 in the openings 42.

In the illustrative embodiments shown, two openings 12, 32, 43 are each provided in a holder 1, 3, 4. This number has proven particularly advantageous for practical implementation, since most conditions arising during the operation can be covered by two different heights. However, this is not intended to rule out the possibility of holders being provided with another number of openings. A design with only one opening 32 may be recommended in particular for holders 3 which are produced as disposable holders from deep-drawn web material 30.

1. An arrangement comprising a holder configured for holding intervertebral prostheses, at least two intervertebral prostheses of different widths which each consist of two end plates with ventral flange flanges and a joint core arranged between the end plates,
the holder comprising a support plate with at least one opening formed therein the opening being configured to receive one of the intervertebral prostheses and having a width which is greater than the maximum width of the intervertebral prostheses,
wherein a pair of steps is formed on at least one longitudinal edge of the opening and their the steps having flanks directed toward one another and spaced apart such that the flange of the intervertebral prosthesis inserted into the opening is centered in the opening.

2. The arrangement as claimed in claim 1, wherein the pair of steps is configured as projections.

3. The arrangement as claimed in claim 1, wherein the pair of steps is configured as depressions.

4. The arrangement as claimed in claim 1, 2, or 3, wherein the flanks taper conically toward one another, the spacing between the flanks decreasing toward the support plate.

5. The arrangement as claimed in claim 1, 2 or 3, wherein a spacing between the flanks is such that, at a deepest point of the flanks matches the width of the flange.

6. The arrangement as claimed in claim 1, wherein the support plate further comprises top parts with a guide.

7. The arrangement as claimed in claim 6, wherein the guide is designed as a channel with a U-shaped cross section.

8. The arrangement as claimed in claim 6 or 7, wherein the top parts are configured in one piece with the pair of steps.

9. The arrangement as claimed in claim 1, 2 or 3, wherein the opening is closed at the bottom.

10. The arrangement as claimed in claim 1, 2 or 3, wherein the support plate is formed in one piece with a substructure.

11. The arrangement as claimed in claim 1, 2 or 3, wherein the support plate is shaped from a web material.

12. The arrangement as claimed in claim 11, wherein the web material is deep-drawn.

13. The arrangement as claimed in claim 1, 2 or 3, wherein the opening is closed by means of a cover.

14. The arrangement as claimed in claim 4, wherein a spacing between the flanks is such that, at a deepest point of the flanks matches the width of the flange.

15. The arrangement as claimed in claim 1, wherein the flanks taper conically toward one another, the spacing between the flanks decreasing toward the support plate, and the support plate further comprises top parts with a guide.

16. The arrangement as claimed in claim 15, wherein the guide is designed as a channel with a U-shaped cross section.

17. The arrangement as claimed in claim 15 or 16, wherein the top parts are configured in one piece with the pair of steps.

18. The arrangement as claimed in claim 1, wherein the flanks taper conically toward one another, the spacing between the flanks decreasing toward the support plate and the opening is closed at the bottom.

19. The arrangement as claimed in claim 18, wherein the support plate is formed in one piece with a substructure.

20. The arrangement as claimed in claim 18, wherein the support plate is shaped from a web material.

21. The arrangement as claimed in claim 20, wherein the web material is deep-drawn.

22. The arrangement as claimed in claim 18, wherein the opening is closed by means of a cover.
